(19) United States
(12) Patent Application Publication
(10) Pub. No.: US 2011/0053208 A1
(43) Pub. Date: Mar. 3, 2011

(54) BIOLOGICAL SAMPLE IDENTIFICATION SYSTEM

(75) Inventors: Rebecca Anne Reiss, Omaha, NE (US); Matthew Robert Kreifels, Omaha, NE (US); David Lee Oliver, Weeping Water, NE (US)

(73) Assignee: Streck, Inc., LaVista, NE (US)

(21) Appl. No.: 12/871,955

(22) Filed: Aug. 31, 2010

Related U.S. Application Data
(60) Provisional application No. 61/238,327, filed on Aug. 31, 2009.

(51) Int. Cl. C12Q 1/02; B01L 9/00

(52) U.S. Cl. 435/29; 422/561; 53/397

(57) ABSTRACT

The present invention contemplates a handling device for a biological specimen. The handling device includes a substrate with at least one crease that divides the substrate into a handle portion and a receiving portion that includes an aperture having a perimeter that is configured so that it receives a container having a cover that includes a biological specimen for testing, while resisting pull-through of the container relative to the substrate.

### FIELD OF THE INVENTION

This invention relates to the handling of labeled articles and more particularly to the handling of labeled biological specimens.

### BACKGROUND OF THE INVENTION

The labeling of biological specimens presents a number of challenges for diagnostic laboratories. It is imperative that appropriate patient identification data is maintained with a biological specimen to avoid the undesirable consequence of providing an inaccurate diagnosis to a patient. Further, the patient identification information must follow every specimen that is transferred from an original specimen container, leaving additional room for error in transferring the identification information accurately. Further, many labeling techniques can make processing of the samples difficult, especially when processing requires visual detection of a biological sample within a container or requires that the sample be placed in a testing device having no additional space for container attachments. Labels that cover some or all of a specimen container make it difficult to process the sample as needed. Therefore, there is a need for devices that improve the labeling and handling of biological specimens.

U.S. Pat. No. 6,860,513 discloses a label for use on a medical container including an adhesive component and a silicone liner component. The label contains printed information and is large enough to substantially wrap around the medical container while a portion of the label extends radially from the surface of the medical container. The label also covers the outer surface of the medical container.

U.S. Pat. No. 4,513,522 discloses a label for a laboratory specimen container that facilitates the attachment of an additional label element to the container. The label uses an adhesive for attachment to the specimen container and a flexible member for attaching to the additional label element. The label covers a portion of the specimen container.

U.S. Pat. No. 5,490,658 discloses a label hangar assembly for use on intravenous bottles. The assembly includes a label sheet having a label portion and hang tabs, and an adhesive located on the label portion but not the hang tabs. The label portion is attached to an I.V. bottle via the adhesive and the hang tabs are partially separated from the label sheet for attachment to an I.V. stand. The label covers a portion of the I.V. bottle.

U.S. Pat. No. 5,468,022 discloses a sample tube identification flag including an elongated portion and multiple connecting portions for attaching the elongated portion to the sample tube. While the arrangement of the connecting portions allows for the tube to be identified without covering a substantial portion of the tube, the shape of many testing devices may require that the label be removed to facilitate accurate testing.

U.S. Pat. No. 3,994,085 discloses a baggage tag adapted to be looped through a luggage or bag handle. The tag is formed of a single continuous sheet of material and folded so that a loop is formed. The tag, however, includes no means for attaching to a medical container that does not have a handle.

Notwithstanding the above, there remains a need for labeling and handling devices that are simple and easily applied and can provide secure identification information for biological specimen containers. There is a further need for labeling and handling devices that do not interfere with testing devices by obscuring a portion of the specimen container or preventing proper fit within a testing device.

### SUMMARY OF THE INVENTION

The present invention contemplates a handling device for a biological specimen comprising a substrate including at least one crease that divides the substrate into a handle portion having at least one peripheral edge portion and a receiving portion that includes at least a portion of an aperture having a perimeter that is configured so that it receives a container having a cover that includes a biological specimen for test, and resists pull-through of the container relative to the substrate. The substrate initially has a substantially flat profile and in use is folded about the crease so that the handle portion and the receiving portion are at an angle other than about 180° relative to each other, and a user can transport the container using the handle portion without directly contacting or otherwise gripping the container.

The present invention further contemplates a method of using the handling device that includes the steps of collecting a plurality of biological test specimens in their own respective individual containers, each container having a closed end and an end including a cover. The method further includes providing a handling device for each biological test specimen wherein the handling device comprises a substrate including at least one crease that divides the substrate into a handle portion having at least two peripheral edge portions and a receiving portion that includes at least a portion of an aperture Each specimen may then be placed through the receiving portion so that an edge of the portion of an aperture contacts the cover of the container thereby resisting pull-through of the container relative to the substrate. The substrate may be folded along the at least one crease so that the peripheral edge portions contact one another and the handle portion and the receiving portion are at an angle other than about 180° relative to each other. The peripheral edge portions may then be secured so that they maintain contact with one another and so that a user can transport the container using the handle portion without directly contacting or otherwise gripping the container.

The present invention also contemplates a method of performing erythrocyte sedimentation rate analysis on a biological specimen. The method includes the steps of drawing a biological specimen from an individual directly into an open end of a specimen container, placing a closure device onto the open end of the specimen container so that the biological specimen remains inside of the specimen container, and placing the specimen container into a handling device. The handling device may include at least one crease that divides the substrate into a handle portion and a receiving portion that includes an aperture so that the specimen container is placed at least partially through the aperture. The handling device may then be folded along the at least one crease so that the handle portion and the receiving portion are at an angle other than about 180° relative to each other and the specimen container may then be placed into an automated erythrocyte sedimentation rate analyzer so that the substrate remains above the specimen container and outside of the analyzer.

The present invention provides a number of benefits, including allowing for a specimen container to be properly labeled and handled so that: the label does not separate from the specimen; the label and handling device does not cover the outer surface of the container so as to avoid interference with visual testing; the location of the label and handling device does not prevent a specimen container from fitting properly within a testing device; and the specimen container can be handled and transported without direct contact with the container.

### BRIEF DESCRIPTION OF THE DRAWINGS

FIG. 1 is a view of a plurality of attached handling devices of the present invention.

FIG. 2 is a perspective view of the invention.

FIG. 3 is a view of the invention prior to insertion of a specimen container.

FIG. 4 is a view of the invention after insertion of a specimen container and including a label.

FIGS. 5A, 5B and 5C are views of an additional embodiment of the invention.

### DESCRIPTION OF THE PREFERRED EMBODIMENT

The present invention provides a unique approach for the identification and handling of packaged materials and particularly biological materials that are obtained from a patient or other individual and placed within a specimen container. The handling device of the present invention uses minimal material and is easily applied to a specimen container while still providing identification information that is securely attached and yet avoids any obfuscation of the biological materials within the labeled container. A number of biological specimen testing and analyzation processes require visual readings of the contents of a specimen container. As a non-limiting example, erythrocyte sedimentation rate (ESR) analysis requires measurement of the sedimentation rate of aggregated erythrocytes in plasma. The rate of sedimentation is an indirect means of analyzing Rouleaux formation and aggregation of erythrocytes (red blood cells). The rate of sedimentation is either measured manually or via an automated instrument, but either process requires visual detection of the levels within the specimen container. Further, many machines that perform automated analyses of any kind require that a specimen container be placed into a space that is designed for close or even friction fit with a specimen container. Thus, it is important that the substrate of the present invention can be attached to a specimen container so that it remains outside of an automated machine during specimen analyzation. The handling device of the present invention allows for appropriate labeling of a specimen container while allowing the contents of the container to be viewed substantially in their entirety. Examples of automated processors of ESR data are sold by Streck, Inc. of Omaha, Nebr. under the name ESR-100 and ESR Auto-Plus.

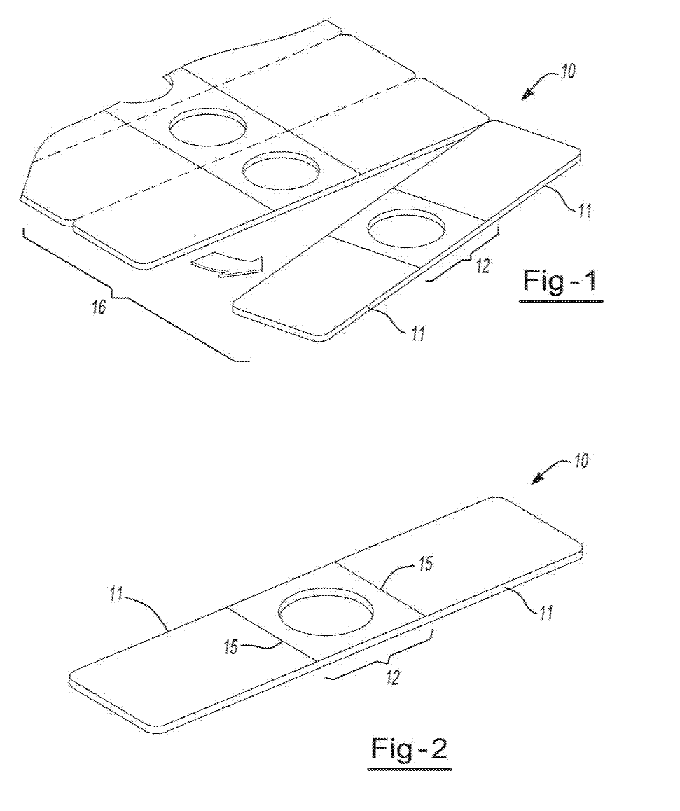

As illustrated (for example) in FIGS. 1 and 2, the invention employs an elongated substrate 10 that includes one or more handle portions 11 and at least one receiving portion 12. The substrate 10 may be substantially flat prior to use. Preferably, the receiving portion 12 includes an aperture through which a specimen container may be placed. In order to maintain the specimen container within the aperture, the perimeter of the aperture may be arranged so that only a portion of a specimen container can fit within the confines of the aperture while an additional portion of the specimen container cannot fit within the aperture to prevent pull-through of the specimen container relative to the substrate. That is, the size of the aperture when applied to the container is smaller than a dimension (e.g. a generally transverse cross-sectional area) of the container. The specimen container may include a cap, cover or lid portion having dimensions that are larger than those of the aperture so that the cap, cover or lid portion cannot be pulled through the aperture, while the dimensions of the remainder of the specimen container are smaller than those of the aperture and so can be pulled through the aperture.

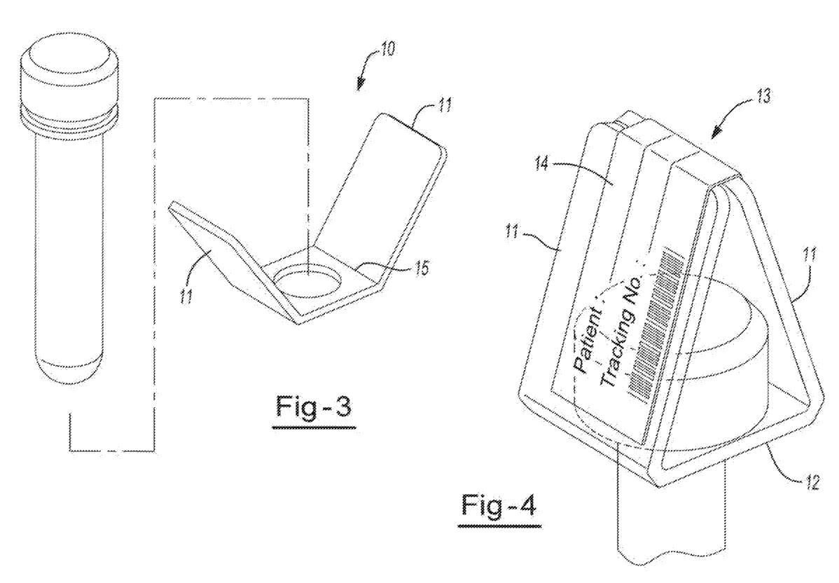

As illustrated in FIGS. 3 and 4, during use, the substrate 10 is folded so that the handle portions 11 are brought toward each other, e.g., they are placed into contact with one another, or nearly in contact with one another, to form an open or closed end loop structure 13. In order to connect the handle portions, one or more of the handle portions may include an adhesive or other means for fastening the handle portions together. The handle portions may also be stapled or taped together. The handle portions may be connected via an additional connection piece. The loop structure 13 may allow for transport of a specimen container via the loop so that the specimen container can be transported while only contacting the handling device with no contact of the specimen container. The user can handle the container without grabbing the container (e.g. without the user's fingers touching the container directly or via gloves). The handle portions may be slit to receive one another. The loop structure 13 further helps to maintain the specimen container within the aperture of the receiving portion. Without the loop structure, the handling device could easily become disengaged from the specimen container.

The loop structure 13 may be further configured to allow for top-access into a specimen container. During specimen processing, a sample may need to be accessed to transfer a portion of the specimen to a new container or to add additional processing reagents to a specimen. In one embodiment, the substrate may contain more than one aperture for entry of the specimen container so that the loop structure will not cover the top portion of the specimen container. In another embodiment, the substrate material may be sufficiently flexible and lengthy so that it can be pulled to the side of the specimen container during processing. In yet another embodiment, the substrate and aperture may be arranged so that the handle portions do not intersect directly above the top portion of the specimen container. The handle portions may not intersect at all and the substrate may be held in place by a fastening mechanism. Also, the aperture may include small slits that allow for expansion and contraction of the aperture size so that upon contraction the handling device is held in place so that the handle portions do not need to intersect. In another embodiment, the aperture may include an adhesive that maintains the handling device in contact with the specimen container, again removing the need for connection of the handle portions.

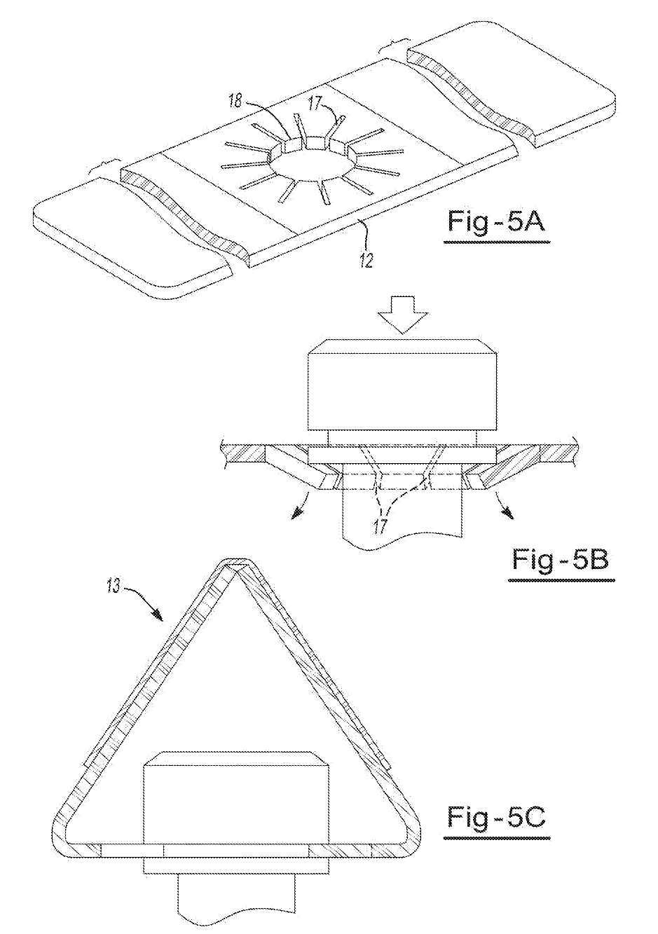

As shown in FIGS. 5A, 5B and 5C, the perimeter of the aperture may include means by which the aperture can be expanded and/or contracted so that the aperture is large enough in its expanded state for a specimen container to be placed within the aperture, but can also be made small enough in its contracted state to hold the specimen container within the desired confines of the aperture to prevent pull-through of the specimen container relative to the substrate. The means for allowing expansion and contraction of the aperture may include a plurality of small incisions or slits 17 located about the perimeter 18 of the aperture. These incisions or slits 17 may thus allow the size of the perimeter of the aperture to change and adjust according to the size and shape of the specimen container. The ability of the aperture to contract to the appropriate specimen container size may also aid in holding the handling device in place.

A label 14 or other additional component may be placed in contact with the handle portions so that the contact or near contact of the handle portions is securely maintained. The label 14 may include a fastening means for securing the label to the handle portions. The fastening means may be an adhesive or may be a mechanical fastening means such as a clip. The fastening means may also be magnetic or elastic in nature. The label may also be friction fit onto the handle portions. The label may be of appropriate dimensions so that it fits within the edges of the handle portions. The label may also be large enough to extend beyond the edge of the handle portions. Preferably, the two handle portions are connected to one another at their terminating ends, but the handling device could be arranged so that the terminating end of one handle portion connects to any area of another handle portion, or any area of one handle portion could connect to any area of another handle portion.

The label 14 may be blank or may include identifying information. The label may include data regarding the origin of the specimen or data regarding the tests to be performed on the specimen. The label may only include a code or the like so that private patient information is not displayed on the label. Identifying information may be pre-printed onto the label, or the label may include space for information to be placed onto the label after the label is attached to the handling device. The label may also include both pre-printed information and room for additional information to be added after attachment to a specimen container. The label, the substrate, or both may include an RFID device to aid in identifying a specimen.

The substrate may include one or more creases 15 at which points the substrate can be easily folded without damage to or breaking of any portion of the handling device. The one or more creases 15 can be placed at any point along the substrate, so long as they avoid contact with the perimeter of the aperture, or are offset from the perimeter of the aperture. The substrate may include two creases that are located on either side of the aperture between the perimeter of the aperture and the ends of the substrate. The one or more creases may be arranged perpendicular to the length (I) of the substrate and extend from one edge of the substrate to the opposing edge of the substrate. The one or more creases may be arranged substantially parallel to one another. The substrate may also include two substantially parallel opposing creases that each define one edge of two handle portions. The one or more creases may also be arranged at a non-perpendicular angle to the length (I) of the substrate so that when the handle portions are placed into contact with one another it is still possible to access the top portion of the specimen container.

The substrate may be composed of a single material or of multiple materials. The substrate can be constructed of any number of materials including plastic, natural fibers, paper, cardboard, foil, film, rubber, foam or any combination thereof. The substrate may be composed of a thermoplastic, formed by extrusion that is subsequently cut into the appropriate arrangement. The substrate may be transparent or opaque or any combination thereof. The material used for construction of the substrate generally is thick and/or strong enough so as to avoid breakage, but thin enough that the substrate is allowed to bend and fold. Therefore, the thickness of the substrate depends on the type of material used to construct the substrate. The substrate may be a thermoplastic material having a thickness of about 0.01 to about 1 mm (e.g., the thermoplastic substrate has a thickness of about 0.05 to about 0.6 mm). The substrate may be composed of a material that can be easily written upon or may include a coating that can be easily written upon. The substrate may also include a release liner material so that information can be applied to the substrate in a removable and/or transferable manner, such that a releasable portion of the substrate can be removed or added via a liner material.

The length of the substrate can vary substantially but should not be too long so as to make the handling of the specimen container difficult. The length of the substrate may be about 30 to about 200 mm (e.g., the length of the substrate may be about 60 to about 120 mm). The width of the substrate can also vary but should not be too wide to make handling of the specimen container difficult or to prevent insertion of the specimen container into any type of testing, carrying or storage device. The width of the substrate may be about 0.5 to about 40 mm (e.g., the width of the substrate is about 10 to about 25 mm). The substrate may have a length that is greater than its width, such that the ratio of length to width is from about 1.5:1 to about 8:1 (e.g., from about 2:1 to about 4:1). The width of the substrate may also be greater than the length of the substrate.

As shown in FIG. 1, multiple handling devices may be included in one multi-substrate sheet 16. Each substrate may be surrounded by perforations to allow for removal of each separate substrate. The multi-substrate sheet may be composed of the same material as the substrate but could also be composed of a different material. The handling devices may also be individually packaged, or packaged in a strip format such that each handling device is connected to another handling device along one or both of its shorter ends. The handling devices may also be packaged in a stackable manner where the handling devices may or may not be connected to one another.

The handling devices may also be designed to withstand at least one use, such that the handling device is placed onto a specimen container and discarded once the specimen has been removed from the container or is no longer needed. The handling devices may also be used multiple times. In the event that material from a specimen container is removed from one specimen container and placed in a different container, the handling device could be removed from the original specimen container and placed onto the new specimen container. A handling device could also be simply re-used so long as any information relating to a previously identified specimen has been removed from the handling device.

The handling device may also include an additional attachment portion that may be connected to the handling device via any connection mechanism or may be integrally formed as part of the handling device. The additional attachment portion may allow for the handling device to be connected to or placed within a transport or storage location. The additional attachment portion may be a hook or loop shaped attachment that is removable from the handling device or integrally formed with the handling device. The additional attachment portion may be used to connect the handle portions of the handling device. The additional attachment portion may be formed to allow simplified carrying of the specimen container by hand while preventing contact with the specimen container, the handling device itself, or both.

The handling device may also be employed in conjunction with a blood screening device such as that disclosed in U.S. Publication No. 2010/0167271 filed with the USPTO on Dec. 23, 2009, and incorporated by reference herein for all purposes. The blood screening device includes a receptacle that receives a sample of blood and that is substantially transparent over at least a portion of its area. The device includes a first end, a second end, a base portion located a distance between the first end and second end that divides the receptacle into a receiving portion and an elongated channel portion, wherein the first end and second end are both open. The device further includes a screening preservative composition placed within the receptacle and being visible through the substantially transparent window, the screening preservative composition being in solid form and located in the top portion of the receptacle and being of sufficient concentration so that upon contact with the sample of blood the screening preservative composition will disperse in the sample, and substantially preserve white blood cell components in the sample. The handling device may be employed with existing safety coated tubes such as those disclosed in U.S. Pat. Nos. 7,419,832 and 7,767,460, incorporated by reference herein for all purposes.

The handling device may also be part of a kit that contains one or more handling devices and one or more labels for attachment to each handling device. The kit may further include one or more specimen containers.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of, or even consist of, the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps. Likewise, any reference to “first” or “second” items is not intended to foreclose additional items (e.g., third, fourth, or more items); such additional items are also contemplated, unless otherwise stated.

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints. The specification of ranges herein also contemplates individual amounts falling within the range. Thus, for example, a range of 10 to 15 contemplates individually the amounts of 10, 11, 12, 13, 14, and 15.

Unless stated otherwise, dimensions and geometries of the various structures depicted herein are not intended to be restrictive of the invention, and other dimensions or geometries are possible. Plural structural components can be provided by a single integrated structure. Alternatively, a single integrated structure might be divided into separate plural components. In addition, while a feature of the present invention may have been described in the context of only one of the illustrated embodiments, such feature may be combined with one or more other features of other embodiments, for any given application. It will also be appreciated from the above that the fabrication of the unique structures herein and the operation thereof also constitute methods in accordance with the present invention.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

1. A handling device for a biological specimen comprising:
a substrate including:
a) at least one crease that divides the substrate into:
i. a handle portion having at least one peripheral edge portion;
ii. a receiving portion that includes at least a portion of an aperture having a perimeter that is configured so that it receives a container having a cover that includes a biological specimen for test, and resists pull-through of the container relative to the substrate;
wherein the substrate initially has a substantially flat profile and in use is folded about the crease so that the handle portion and the receiving portion are at an angle other than about 180° relative to each other, and a user can transport the container using the handle portion without directly contacting or otherwise gripping the container.

2. The handling device of claim 1, wherein the crease of the substrate is laterally offset relative to the perimeter of the aperture.

3. The handling device of claim 1, wherein the substrate includes a pair of substantially parallel creases, defining at least two handle portions.

4. The handling device of claim 1, wherein the substrate includes a pair of substantially parallel opposing creases external of the perimeter of the aperture and on either side for defining two handle portions.

5. The handling device of claim 1, wherein the perimeter of the aperture is configured so that its perimeter dimensions are larger than that of one of the container or cover, and smaller than that of the other of the container or cover.

6. The handling device of claim 1, wherein the handle portion includes an adhesive in contact therewith.

7. The handling device of claim 1, wherein the handle portion includes an adhesive label thereon.

8. The handling device of claim 1, wherein the handle portion includes an adhesive label that extends beyond the at least one peripheral edge.

9. The handling device of claim 1, wherein the substrate includes a material selected from paper, cardboard, foil, film, or any combination thereof.

10. The handling device of claim 1, wherein an adhesive label connects at least two handle portions with each other.

11. The handling device of claim 1, wherein the device initially has a substantially flat profile and in use is folded about two creases so that a pair of opposing handle portions project generally away from the receiving portion and are connected to each other by an adhesive label.

12. A method of handling biological test specimens comprising the steps of:
a) collecting a plurality of biological test specimens in their own respective individual containers, each container having a closed end and an end including a cover;
b) providing a handling device for each biological test specimen wherein the handling device comprises a substrate including at least one crease that divides the substrate into a handle portion having at least two peripheral edge portions and a receiving portion that includes at least a portion of an aperture;
c) placing each biological test specimen through the receiving portion so that an edge of the portion of an aperture contacts the cover of the container thereby resisting pull-through of the container relative to the substrate;
d) folding the substrate along the at least one crease so that the peripheral edge portions contact one another and the handle portion and the receiving portion are at an angle other than about 180° relative to each other;
e) securing the peripheral edge portions so that they maintain contact with one another and so that a user can transport the container using the handle portion without directly contacting or otherwise gripping the container.

13. The method of claim 12, wherein the peripheral edge portions are adhered to one another by an adhesive label portion.

14. The method of claim 12, wherein the crease of the substrate is laterally offset relative to the perimeter of the aperture.

15. The method of claim 12, wherein the diameter of the cover is greater than the diameter of the aperture and the diameter of the container is less than the diameter of the aperture.

16. The method of claim 15, wherein each specimen container is placed through the aperture by placing the closed end of the container through the aperture first and the aperture is maintained just below the cover due to the diameter of the cover being greater than the diameter of the aperture.

17. The method of claim 12, wherein the substrate includes a pair of substantially parallel creases, defining at least two handle portions.

18. The method of claim 13, wherein the adhesive label located in contact with the substrate is the only label so that no label is placed directly onto the container.

19. The method of claim 12, wherein after securing the peripheral edge portions, a user can transport the container using the handle portion without directly contacting or otherwise gripping the container.

20. A method of performing erythrocyte sedimentation rate analysis on a biological specimen comprising:
a) drawing a biological specimen from an individual directly into an open end of a specimen container;
b) placing a closure device onto the open end of the specimen container so that the biological specimen remains inside of the specimen container;
c) placing the specimen container into a handling device, wherein the handling device includes:
i. at least one crease that divides the substrate into a handle portion and a receiving portion that includes an aperture so that the specimen container is placed at least partially through the aperture;
d) folding the substrate along the at least one crease so that the handle portion and the receiving portion are at an angle other than about 180° relative to each other;
e) placing the specimen container into an automated erythrocyte sedimentation rate analyzer so that the substrate remains above the specimen container and outside of the analyzer.
